(19) United States
(12) Patent Application Publication
(10) Pub. No.: US 2025/0302531 A1
(43) Pub. Date: Oct. 2, 2025

(54) DEVICES, SYSTEMS, AND METHODS FOR ULTRASOUND THERAPY

(71) Applicant: University of Kansas, Lawrence, KS (US)

(72) Inventors: Xinmai Yang, Lawrence, KS (US); M. Laird FORREST, Lawrence, KS (US)

(73) Assignee: University of Kansas, Lawrence, KS (US)

(21) Appl. No.: 18/865,299

(22) PCT Filed: May 12, 2023
(86) PCT No.: PCT/US2023/022119

§ 371 Date: Nov. 12, 2024

Related U.S. Application Data
(60) Provisional application No. 63/341,624, filed on May 13, 2022.

(51) Int. Cl. A61B 18/24; A61B 17/22; A61B 17/225; A61B 18/00

(52) U.S. Cl. CPC A61B 18/245; A61B 17/225; A61B 2017/22058; A61B 2018/00994

(57) ABSTRACT

A system may include an ultrasound transducer. A system may include a catheter for insertion into a body lumen of a patient, the catheter including a distal end. A system may include a reflector connected to the distal end of the catheter, the reflector formed from a material having an acoustic impedance mismatch with an adjacent material. Optical energy may be delivered through the catheter.

### CROSS-REFERENCE TO RELATED APPLICATIONS

This application claims priority to and the benefit of U.S. Provisional Patent Application No. 63/341,624, filed on May 13, 2022, which is hereby incorporated by reference in its entirety.

### GOVERNMENTAL RIGHTS

This invention was made with government support under grant no. HL147783, HL152410, and EY029489 awarded by the National Institutes of Health. The government has certain rights in the invention.

### BACKGROUND

Atherosclerosis is a medical condition in which arteries harden and narrow due to the buildup of fat, cholesterol, calcium and other substances on the inner artery walls. Atherosclerosis is the major cause of cardiovascular disease such as ischemic heart disease and ischemic stroke, and atherosclerotic cardiovascular disease (ACD) is a major cause of death in the United States (US) and worldwide. The direct and indirect cost of ACD in 2017-2018 was $229 billion in the US.

Several medications can slow down or reverse the process of atherosclerosis. Cholesterol lowering medications, blood thinners and blood pressure medications are the most commonly used. Cholesterol lowering medications such as statins slow down the buildup of lipids in the arteries by reducing low-density lipoprotein cholesterol in the blood. Blood thinners such as aspirin are often used to prevent the formation of blood clots. Blood pressure medications are used to reduce the risk of plaque rupture by controlling high blood pressure. If medications are not effective in treating the atherosclerotic plaque blockages, interventional surgeries may be performed on the affected portion of the arteries. The most common surgical procedure for coronary atherosclerosis is percutaneous coronary intervention (PCI), also known as angioplasty and stenting. In case of atherosclerosis in the carotid artery, the plaque buildup is more often removed surgically by a procedure known as endarterectomy, which is more effective than carotid angioplasty and stenting. For severe atherosclerosis in coronary arteries, also known as multi-vessel disease, a coronary artery bypass grafting can be used to create a bypass and redirect the blood flow around the blocked artery.

Since the 1990s, laser technology has been used to modify atherosclerotic plaques during PCI, a technique commonly known as excimer laser coronary angioplasty (ELCA).

This technique uses a nanosecond (125-200 ns) pulsed laser in the ultraviolet range (100-400 nm) to remove plaques by photochemical, photothermal and photomechanical mechanisms. ELCA uses a high laser fluence in the range of 30-80 mJ/mm² and a pulse repetition frequency of 25-80 Hz. However, the use of high laser fluence and pulse repetition frequency results in complications, such as an increased risk of vessel dissection and perforation.

In some situations, atherosclerotic plaques may be disrupted using a combined ultrasound and laser irradiation to modify the plaque by enhancing cavitation. This technique may be referred to as ultrasound-assisted endovascular laser thrombolysis (USELT), which removes blood clots using combined ultrasound and laser. The combination of ultrasound and laser results in enhanced cavitation, which precisely disrupts and breaks the blood clot without causing any damage to the nearby tissues.

### BRIEF SUMMARY

In some aspects, the techniques described herein relate to a system. The system includes an ultrasound transducer. A catheter is configured for insertion into a body lumen of a patient. The catheter includes a distal end. A reflector is connected to the distal end of the catheter. The reflector is formed from a material having an acoustic impedance mismatch with an adjacent material.

In some aspects, the techniques described herein relate to a method. The method includes transmitting ultrasound energy into a plaque deposit in a body lumen. At least a portion of the ultrasound energy is reflected back to said plaque deposit with a reflector, the reflector connected to a distal end of a catheter located at said plaque deposit in said body lumen.

In some aspects, the techniques described herein relate to a method. The method includes positioning an acoustic energy source outside of a patient's body and focused at a target location in said patient's body. The target location includes a plaque deposit. A reflector connected to a distal end of a catheter is positioned proximate said plaque deposit at the target location. Ultrasound energy is transmitted to the reflector, the reflector reflecting at least a portion of the ultrasound energy to said plaque deposit.

This summary is provided to introduce a selection of concepts that are further described below in the detailed description. This summary is not intended to identify key or essential features of the claimed subject matter, nor is it intended to be used as an aid in limiting the scope of the claimed subject matter. Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a perspective view of an embodiment of a system for removing plaque accumulations in a body lumen, according to at least one embodiment of the present disclosure;

FIG. 2 is a schematic representation of a plaque-removal system, according to at least one embodiment of the present disclosure;

FIG. 3 is a schematic representation of a plaque-removal system, according to at least one embodiment of the present disclosure;

FIG. 4 is a representation of a plaque-removal system, according to at least one embodiment of the present disclosure;

FIG. 5 is a representation of a plaque-removal system, according to at least one embodiment of the present disclosure;

FIG. 6 is a representation of a plaque-removal system, according to at least one embodiment of the present disclosure;

FIG. 7 is a flowchart of a method for removing plaque, according to at least one embodiment of the present disclosure; and

FIG. 8 is a flowchart of a method for removing plaque, according to at least one embodiment of the present disclosure.

### DETAILED DESCRIPTION

This disclosure generally relates to devices, systems, and methods for removing plaque deposits in a patient's blood vessels. More particularly, the present disclosure relates to the use of a reflector to reflect ultrasonic energy toward the plaque deposits for thrombolysis for removing blood clots in deep vessels. In some embodiments, an endovascular laser may be combined with the ultrasonic energy to further facilitate removal of the plaque deposits. In some embodiments, systems and methods of photo-mediated ultrasound therapy (PUT) described in the present disclosure may allow blood clot removal with increased precision and/or selectivity when compared to conventional therapies. Indeed, the use of the reflector to reflect the ultrasonic energy back to the plaque deposits may help to reduce the total amount of ultrasonic energy applied to the patient's body. This may help to reduce the energy input of the ultrasonic energy used in the PUT, thereby reducing or preventing damage to the patient's body from the ultrasonic energy.

PUT includes a combined use of optical energy and acoustic energy to selectively target specific tissue or material in a body. The increased selectivity of PUT compared to conventional therapies may be at least partially related to the endogenous optical contrast between tissue types. Different tissues in an organism contain different concentrations of various chromophores and have different optical absorption spectra. The optical absorption spectra can facilitate the differentiation of tissue types with multi-spectral optical techniques that are highly sensitive and specific. PUT has the unique capability to target, without any exogenous agent, blood clots by taking advantage of the high native contrast in the optical absorption between different other tissues. For example, hemoglobin absorbs more optical energy than other tissues at certain wavelengths, and excitation (and cavitation) can be limited to the blood vessel, providing highly localized treatment. In contrast to conventional therapeutic techniques utilizing ultrasound contrast agents and/or nanoparticles to catalyze cavitation, PUT may produce cavitation in the targeted blood clot or other tissue without additional agents and selectively at the confluence of the applied optical energy and acoustic energy.

The combination of the optical energy and acoustic energy may produce photospallation. Photospallation is the creation of thermoelastic stress in the blood clot via a photoacoustic effect. Upon application of the optical energy, a photoacoustic wave is produced in or on a surface of the blood clot. The oscillating cavitation may then apply mechanical stresses to the blood clot producing thrombolysis.

The PUT techniques described herein combining acoustic energy and optical energy are more precise and require less total energy to be applied to the patient's tissue than conventional acoustic thrombolysis techniques or conventional optical thrombolysis techniques alone. Conventional acoustic thrombolysis uses much higher negative pressures between 14 MPa and 19 MPa (such as described in Maxwell et al., “Noninvasive treatment of deep venous thrombosis using pulsed ultrasound cavitation therapy (histotripsy) in a porcine model”, J Vasc Interv Radiol. 2011 March; 22(3): 369-377. doi: 10.1016/j.jvir.2010.10.007) compared to acoustic pressures of less than 5 MPa, as described according to the present disclosure. Conventional optical thrombolysis uses much higher optical energy with greater fluence of 30 to 80 mJ/mm² and pulse lengths of 185 ns (such as described in Nagamine et al., “Comparison of 0.9-mm and 1.4-mm catheters in excimer laser coronary angioplasty for acute myocardial infarction”, Lasers in Medical Science (2019) 34:1747-1754. Doi.org/10.1008/s10103-019-02772) compared to pulse lengths up to 10 ns and fluence up to 500 mJ/cm² (5 mJ/mm²).

Even the combined acoustic energy and optical energy applied by the systems and methods according to the present disclosure is less than the energies applied by the conventional techniques. As described herein, acoustic transducers can have a spot size much larger than the targeted thrombus, resulting in potential damage to the surrounding tissue. Optical conduits increase in diameter according to increases in optical power needed for the application. Therefore, lower optical power allows for smaller diameter conduits that can be more flexible, safer, and able to navigate more vasculature of the patient's body than conduits used for conventional laser thrombolysis. For example, conventional excimer laser catheters used for laser thrombolysis have a diameter of 0.9 mm, 1.4 mm, 1.7 mm, 2.0mm, while embodiments of laser catheters according to the present disclosure have diameters less than 750 microns (0.75 mm), including a 400-micron (0.40 mm) diameter laser catheter used during the testing described herein.

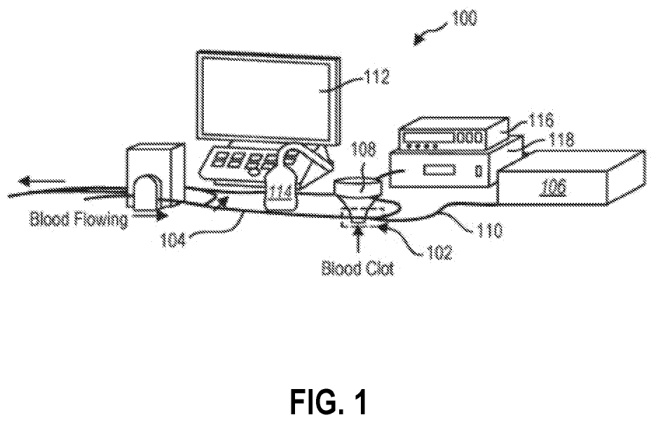

FIG. 1 is a perspective view of an embodiment of a system 100 for removing plaque accumulations in a body lumen, such as by producing cavitation in a vessel. FIG. 1 illustrates a bench test system for providing energies to a target location 102 in a simulated blood vessel 104 with a blood clot in the vessel 104. The system 100 may include an acoustic energy source 108, such as a High-intensity focused ultrasound (HIFU) transducer. The acoustic energy source 108 may be positioned outside of the patient's body. In some embodiments, the HIFU transducer may be directed at the target location 102. For example, the HIFU transducer may be located outside of the vessel 104. The HIFU transducer may have a focus. For example, the HIFU transducer may be focused at the target location 102, such as on a blood clot and/or a plaque buildup.

In accordance with at least one embodiment of the present disclosure, a catheter 110 may be inserted into the vessel 104. The catheter 110 may have a distal end, which may be the end of the catheter 110 that is inserted into the vessel 104. The catheter 110 may include a reflector located in the vessel 104. The reflector may be directed to the target location 102. For example, the reflector may be located proximate to the plaque deposit. In some embodiments, the reflector may be in contact with the plaque deposit.

In accordance with at least one embodiment of the present disclosure, the reflector may reflect at least a portion of the acoustic energy emitted by the acoustic energy source 108. For example, the acoustic waves emitted by the HIFU transducer and focused on the target location 102 may be at least partially reflected by the reflector. In some embodiments, placing the plaque deposit between the reflector and the acoustic energy source 108 may cause the acoustic energy emitted by the acoustic energy source 108 to pass through the plaque deposit, be reflected by the acoustic energy source 108, and pass back through the plaque deposit. In some embodiments, the acoustic energy may combine in the plaque deposit. For example, the reflected sound waves may combine with the inbound sound waves. The combination may cause an increase in the total magnitude of the sound waves. This may increase the energy applied to the plaque deposit to levels above that emitted by the acoustic energy source 108. In this manner, the energy emitted by the acoustic energy source 108 may be reduced to maintain the target level of energy at the target location 102. Reducing the energy applied by the acoustic energy source 108 may help to further reduce the total energy input into the patient at the target location 102, thereby reducing or preventing damage to untargeted tissues. As may be seen in FIG. 1 and the following figures, the HIFU transducer may be separate from the catheter. For example, the HIFU transducer may be located in a different location than the catheter, and the catheter may not include any mechanism to transmit ultrasound energy to the target location.

In some embodiments, the system includes an optical energy source 106, such as a laser source. The laser source may be connected (e.g., coupled) to an optical conduit, such as an optical fiber, located in the catheter 110 to direct the optical energy to the target location 102. In some embodiments, the acoustic energy may not be sufficient to promote cavitation in the vessel 104 individually. In some embodiments, the combination of the acoustic energy and the optical energy promotes cavitation through the synchronized application of the energies. For example, the optical energy and acoustic energy are synchronized such that the optical energy creates a bubble, and the acoustic energy expands or collapses the bubble.

In some embodiments, the acoustic energy is able to penetrate through the surrounding tissue to the target location 102 (e.g., the plaque deposit). The optical energy, however, may have much shorter transmission depths through the tissue, and the optical conduit in the catheter 110 may allow the optical energy to be provided directly to the target location 102 within the vessel 104. The optical conduit in the catheter 110, therefore, may allow PUT in deep vessels, not otherwise accessible from surface application of optical energy. In some embodiments, the optical conduit in the catheter 110 is a fiber optic cannula.

The optical energy source 106 and acoustic energy source 108 may be confocal at the target location 102 (e.g., the energy from both the optical energy source 106 and the acoustic energy source 108 may be focused at the target location 102). In some embodiments, the optical energy source 106 and/or acoustic energy source 108 may be operated in a series of pulses. For example, the optical energy source 106 may be pulsed such that at least a portion of the pulse temporally overlaps with a pulse of the acoustic energy source 108 (when reflected by the reflector). In other examples, the optical energy source 106 may be pulsed during a continuous operation of the acoustic energy source 108. In yet other examples, the optical energy source 106 may be operated continuously while the acoustic energy source 108 is pulsed during the operation of the optical energy source 106.

In some embodiments, the optical energy source 106 and/or the acoustic energy source 108 may be controlled by a computing device 112. In at least one embodiment, the computing device 112 may include or be in communication with a computer readable medium (CRM) that may contain instructions that, when read by the computing device, cause the computing device to perform one or more methods described herein. For example, the timing of the optical energy source 106 pulse and the acoustic energy source 108 pulse may be coordinated by the computing device 112. In other examples, a series of overlapping pulses from the optical energy source 106 and acoustic energy source 108 may be controlled by the computing device 112. In yet other examples, the computing device 112 may be in communication with one or more sensors 114 configured to detect cavitation or other aspects of the target location 102 and/or vessel 104. In such examples, the computing device 112 may pulse the optical energy source 106 and/or acoustic energy source 108 based at least partially on information communicated by the one or more sensors 114.

In some embodiments, the system 100 may include one or more components to provide additional control over the delivery of acoustic energy. For example, the system 100 may include one or more power amplifiers 116 in communication with the acoustic energy source 108. In examples, the system 100 may include one or more function generators 118 in communication with the acoustic energy source 108 to control the phase and/or frequency of the acoustic energy from the acoustic energy source 108.

Acoustic energy may be applied to the target location 102. In some embodiments, an acoustic energy source 108, such as a HIFU transducer or therapeutic ultrasound transducer, may be positioned proximate or adjacent the target location 102. The acoustic energy source 108 may be used to supply ultrasound bursts to the target location 102. As discussed herein, the ultrasound bursts may be reflected through the target tissue with a reflector.

In some embodiments, the acoustic energy source 108 may use an electric potential and/or current to move one or more components of the acoustic energy source 108 and produce an acoustic wave. An annular structure of the acoustic energy source 108 may assist in focusing the acoustic wave through the center of the acoustic energy source 108 and directing the acoustic energy to the target location 102. In some embodiments, the focal length of the acoustic energy source 108 is at least 6 centimeters (cm). In other embodiments, the focal length of the acoustic energy source 108 is less than 10 cm. The focal length of the acoustic energy source 108 may allow the acoustic energy source 108 to focus the acoustic energy at a target location within the patient's tissue while the acoustic energy source 108 is positioned outside of the patient's body.

In some embodiments, the therapeutic ultrasound transducer may provide acoustic energy with a maximum positive pressure and/or maximum negative pressure (below atmosphere) in a range having an upper value, a lower value, or upper and lower values including any of 0.1 Megapascals (MPa), 0.2 MPa, 0.3 MPa, 0.4 MPa, 0.5 MPa, 0.6 MPa, 0.8 MPa, 1.0 MPa, 1.2 MPa, 1.4 MPa, 1.6 MPa, 1.8 MPa, 2.0 MPa, 2.5 MPa, 3.0 MPa, 4.0 MPa, 5.0 MPa, or any values therebetween. For example, the pressure maximum may be greater than 0.1 MPa. In other examples, the pressure maximum may be less than 5.0 MPa. In yet other examples, the pressure maximum may be between 0.1 MPa and 5.0 MPa. In further examples, the pressure maximum may be between 0.2 MPa and 2.5 MPa. In yet further examples, the pressure maximum may be between 0.3 MPa and 2.0 MPa. In at least one example, the pressure maximum may be 0.45 MPa.

In some embodiments, the therapeutic ultrasound transducer may provide acoustic energy with a frequency in a range having an upper value, a lower value, or upper and lower values including any of 500 kHz, 550 kHz, 600 kHz, 650 kHz, 700 kHz, 750 kHz, 800 kHz, 850 kHz, 900 kHz, 950 kHz, 1.0 MHZ, or any values therebetween. For example, the frequency may be greater than 500 kHz. In other examples, the frequency may be less than 1.0 MHz. In yet other examples, the frequency may be between 500 kHz and 1.0 MHz. In further examples, the frequency may be between 550 kHz and 950 kHz. In yet further examples, the frequency may be between 600 kHz and 900 kHz. In at least one example, the frequency may be 750 kHz.

In some embodiments, the duty cycle of the therapeutic ultrasound transducer may

be in a range having an upper value, a lower value, or upper and lower values including any of 0.1%, 1%, 2%, 3%, 4%, 5%, 6%, 7%, 8%, 9%, 10%, or any values therebetween. For example, the duty cycle may be greater than 0.1%. In other examples, the duty cycle may be less than 10%. In yet other examples, the duty cycle may be between 0.1% and 10%. In further examples, the duty cycle may be between 2% and 9%. In yet further examples, the duty cycle of the therapeutic ultrasound transducer may be between 3% and 8% during operation.

In some embodiments, the optical energy source 106 may include one or more lasers. For example, the optical energy source may include an yttrium aluminum garnet (YAG) laser. A neodymium-doped yttrium aluminum garnet (Nd: YAG) pumped optical parametric oscillator (OPO) system may be used as the optical energy source 106 for PUT. The optical energy source 106 may have a peak wavelength in the tuning range. In some embodiments, the peak wavelength may be in a range having an upper value, a lower value, or an upper and lower value including any of 400 nm, 600 nm, 800 nm, 1000 nm, 1200 nm, 1400 nm, 1600 nm, 1800 nm, 2000 nm, 2200 nm, 2400 nm, or any values therebetween. For example, the peak wavelength may be greater than 400 nm. In other examples, the peak wavelength may be less than 2400 nm. In yet other examples the peak wavelength may be in a range of 400 nm to 2400 nm. In further other examples, the peak wavelength may be in a range of 450 nm to 1600 nm. In at least one example, the peak wavelength may be tuned to the peak optical absorption wavelength of the target material, such as hemoglobin.

In some embodiments, an optical power of the optical energy directed at the target location 102 is in a range having an upper value, a lower value, or upper and lower values including any of 1 mW, 2 mW, 4 mW, 6 mW, 8 mW, 10 mW, 15 mW, 20 mW, 40 mW, 60 mW, 80 mW, 100 mW, 200 mW, or any values therebetween. For example, the optical power may be greater than 1 mW. In other examples, the optical power may be less than 200 mW. In yet other examples, the optical power may be between 1 mW and 200 mW. In further examples, the optical power may be between 2 mW and 100 mW. In yet further examples, the optical power may be between 10 mW and 50 mW. In at least one example, the optical power may be 25 mW.

The optical energy source 106 may operate at a repetition rate with a pulse duration. In some embodiments, the optical energy source 106 may be operated at a repetition rate in a range having an upper value, a lower value, or upper and lower values including any of 2 Hz, 4 Hz, 6 Hz, 8 Hz, 10 Hz, 15 Hz, 20 Hz, 40 Hz, or any values therebetween. For example, the repetition rate may be greater than 2 Hz. In other examples, the repetition rate may be less than 40 Hz. In yet other examples, the repetition rate may be in a range of 2 Hz to 40 Hz. In further examples, the repetition rate may be between 6 Hz and 20 Hz. In at least one example, the repetition rate may be 10 Hz.

In some embodiments, the pulse duration may be in a range having an upper value, a lower value, or upper and lower values including any of 0.1 nanosecond (ns), 0.5 ns, 1 ns, 2 ns, 3 ns, 4 ns, 5 ns, 6 ns, 7 ns, 8 ns, 9 ns, 10 ns, or any values therebetween. For example, the pulse duration may be greater than 0.1 ns. In other examples, the pulse duration may be less than 10 ns. In yet other examples, the pulse duration may be between 0.1 ns and 10 ns. In further examples, the pulse duration may be between 2 ns and 6 ns. In at least one example, the pulse duration may be 4 ns.

The optical energy is delivered to the target location 102 and the surface fluence is monitored and controlled during application using the one or more sensors 114 and/or a camera. In some embodiments, the fluence may be in a range having an upper value, a lower value, or upper and lower values including any of 0.1 mJ/cm², 0.5 mJ/cm², 1 mJ/cm², 2 mJ/cm², 4 mJ/cm², 6 mJ/cm², 8 mJ/cm², 10 mJ/cm², 15 mJ/cm², 20 mJ/cm², 40 mJ/cm², 60 mJ/cm², 80 mJ/cm², 100 mJ/cm², 200 mJ/cm², 300 mJ/cm², 400 mJ/cm², 500 mJ/cm², or any values therebetween. For example, the fluence may be greater than 0.1 mJ/cm². In other examples, the fluence may be less than 500 mJ/cm². In yet other examples, the fluence may be between 0.1 mJ/cm² and 200 mJ/cm². In further examples, the fluence may be between 2 mJ/cm² and 100 mJ/cm². In yet further examples, the fluence may be between 3 mJ/cm² and 20 mJ/cm². In at least one example, the fluence may be 4 mJ/cm².

In some embodiments, a laser pulse is delivered to the target location to overlay the rarefaction phase (maximum negative pressure) at the beginning of each ultrasound burst. Timing of the concurrent energy delivery during rarefaction increases the likelihood of cavitation according to the underlying mechanism. In other embodiments, a laser pulse is delivered to the target location to overlay each rarefaction phase (maximum negative pressure) during each ultrasound burst. In some embodiments, the laser pulse is delivered to the target location to overlap with the confluence of incoming and reflected ultrasound pulses reflected from the reflector.

In some embodiments, the treatment duration to remove microvessels in the target area may be in a range having an upper value, a lower value, or upper and lower values of 1 second(s), 5 s, 10 s, 15 s, 30 s, 45 s, 60 s, 75 s, 90 s, 105 s, 120 s, or any values therebetween. For example, the treatment duration may be greater than 1 s. In other examples, the treatment duration may be less than 120 s (2 minutes). In yet other examples, the treatment duration may be less than 90 s. In further examples, the treatment duration may be less than 60 s. In at least one example, the treatment duration may be between 15 s and 90 s.

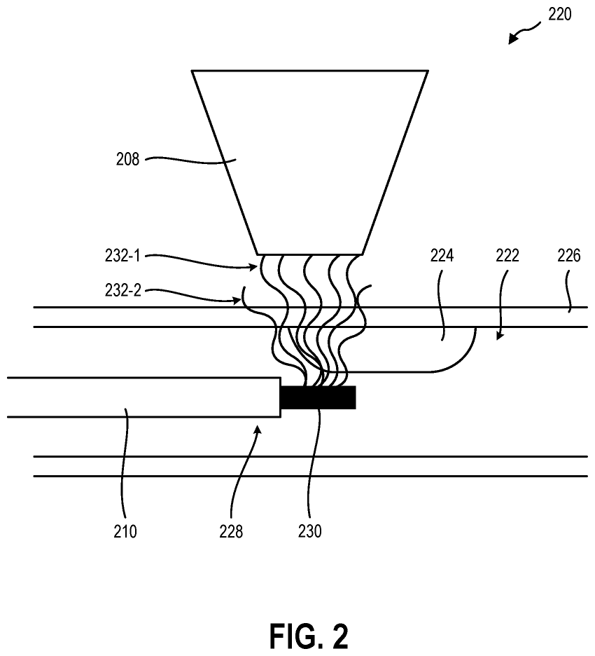

FIG. 2 is a schematic representation of a plaque-removal system 220, according to at least one embodiment of the present disclosure. The plaque-removal system 220 includes an acoustic energy source 208, such as an HIFU transducer. The acoustic energy source 208 may be positioned outside of the patient's body. A body lumen 222, such as the interior of a blood vessel (e.g., an artery, a vein), may include a plaque deposit 224 attached to one of the vessel walls 226. In the embodiment shown, a catheter 210 is inserted into the body lumen 222. A reflector 230 is located at a distal end 228 of the catheter 210 proximate the plaque deposit 224. For example, the reflector 230 may be located longitudinally proximate the plaque deposit 224. In some examples, the reflector 230 may be located such that a plane intersecting the vessel walls 226 and extending into the body lumen 222 may intersect both the plaque deposit 224 and the reflector 230. In some embodiments, the reflector 230 may be radially proximate to the plaque deposit 224. For example, the reflector 230 may be located a reflector distance of the vessel walls 226. The reflector distance may be any value, including 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 4.0 mm, 5.0 mm, or any value therebetween.

The acoustic energy source 208 may emit ultrasound energy (collectively 232) and transmit the ultrasound energy 232 to the target location. The target location may include a location a depth inside the patient's body. The target location may be or include the plaque deposit 224. Put another way, the ultrasound energy 232 may be targeted at a target tissue in a target location. While embodiments of the present disclosure are discussed with respect to a plaque deposit 224 located in a body lumen 222 of a blood vessel, it should be understood that the techniques disclosed herein may include ultrasonic and/or optical energy that is directed to any portions of a user's body, including portions of the stomach, intestines, esophagus, organs, any other portions of a user's body, and combinations thereof.

The acoustic energy source 208 may emit or transmit incoming ultrasound energy 232-1. The incoming ultrasound energy 232-1 may pass through the vessel walls 226 and the plaque deposit 224 toward the reflector 230. When the incoming ultrasound energy 232-1 reaches the reflector 230, the reflector 230 may reflect at least a portion of the incoming ultrasound energy 232-1 as reflected ultrasound energy 232-2. The reflected ultrasound energy 232-2 may be reflected back toward the plaque deposit 224. The reflected ultrasound energy 232-2 may encounter the incoming ultrasound energy 232-1. In some embodiments, the sound waves from the reflected ultrasound energy 232-2 and the incoming ultrasound energy 232-1 may combine amplitude, thereby increasing the total ultrasound energy. In accordance with at least one embodiment of the present disclosure, the combined ultrasound energy may be focused at the plaque deposit 224. In this manner, the energy applied by the acoustic energy source 208 may be increased at the plaque deposit 224. This may allow the incoming ultrasound energy 232-1 emitted or transmitted by the acoustic energy source 208 to be reduced while still applying desired the combined ultrasound energy. As will be understood, the applied combined ultrasound energy may be greater than the incoming ultrasound energy 232-1 and/or the reflected ultrasound energy 232-2.

The reflector 230 may have any shape. For example, the reflector 230 may be cylindrical. In some examples, the reflector 230 may have a conical or frusto-conical shape. In some examples, the reflector 230 may have one or more flat edges. In some examples, the reflector 230 may have one or more concave edges. In some examples, the reflector 230 may have one or more convex edges.

In accordance with at least one embodiment of the present disclosure, the reflector 230 may be formed from a material that is at least partially acoustically reflective. For example, the reflector 230 may be formed from a material that creates an acoustic impedance mismatch. Acoustic impedance is a physical property of a material that is related to how much the material resists the passing of ultrasound waves. A higher acoustic impedance is related to a higher resistance to the passing of ultrasound waves, while a lower acoustic impedance is related to a lower resistance to the passing of ultrasound waves. The acoustic impedance of a material is impacted by various factors, including the physical density of the material and/or the velocity of the acoustic energy.

At the junction between two materials, the ability of the ultrasound wave to pass from one material to the other may be based at least partially on the mismatch of the adjacent materials. For examples, two adjacent materials having a relatively high acoustic impedance mismatch may reflect relatively more ultrasound waves at the junction between the two adjacent materials. Two materials having a relatively low acoustic impedance mismatch may reflect relatively fewer of the ultrasound waves at the junction between the two adjacent materials.

In accordance with at least one embodiment of the present disclosure, the reflector 230 may be formed from a material having a high acoustic impedance. The reflector 230 may be formed from a material that has a higher acoustic impedance than body tissues. For example, the reflector 230 may be formed from a material that has a higher acoustic impedance than a body fluid in the body lumen 222. In some examples, the reflector 230 may be formed from a material that has a higher acoustic impedance than an adjacent tissue, such as the plaque deposit 224 and/or the vessel walls 226. In this manner, when the acoustic energy source 208 emits the incoming ultrasound energy 232-1 toward the reflector 230, the reflector 230 may reflect at least a portion of the incoming ultrasound energy 232-1 back toward the acoustic energy source 208.

In some embodiments, the acoustic impedance mismatch between the reflector 230 and the adjacent material (e.g., the fluid in the body lumen 222, the plaque deposit 224, the vessel walls 226) may have any value. In some embodiments, the acoustic impedance mismatch (e.g., a ratio of acoustic impedance of the reflector 230 to the acoustic impedance of the adjacent material) may be in a range having an upper value, a lower value, or upper and lower values including any of 10:9, 8:7, 7:6, 5:4, 4:3, 3:2, 2:1, 3;1, 4:1, 5:1, 6:1, 7:1, 8:1, 9:1, 10:1, 12:1, 15:1, 20:1, 30:1, 40:1, greater than 40:1, or any value therebetween. For example, the acoustic impedance mismatch may be greater than 10:9. In another example, the acoustic impedance mismatch may be less than 40:1. In yet other examples, the acoustic impedance mismatch may be any value in a range between 10:9 and 30:1. In some embodiments, it may be critical that the acoustic impedance mismatch is greater than 5:1 to reflect a sufficient amount of the incoming ultrasound energy 232-1 to combine to an increase in acoustic energy.

In some embodiments, the reflector 230 may have a reflectivity, which may represent the percentage of the incoming ultrasound energy 232-1 that are reflected into reflected ultrasound energy 232-2. As will be understood, the reflectivity may be related to the acoustic impedance mismatch. In some embodiments, the reflectivity may be in a range having an upper value, a lower value, or upper and lower values including any of 10%, 20%, 30%, 40%, 50%, 60%, 70%, 80%, 90%, 100%, or any value therebetween. For example, the reflectivity may be greater than 10%. In another example, the reflectivity may be less than 100%. In yet other examples, the reflectivity may be any value in a range between 10% and 100%. In some embodiments, it may be critical that the reflectivity is greater than 50% to combine to an increase in acoustic energy.

In some embodiments, the material of the reflector 230 may be any material that has a higher acoustic impedance than the user's body tissues. The material of the reflector 230 may include a metallic alloy, such as a steel alloy, a composite material, a plastic material, any other material, and combinations thereof.

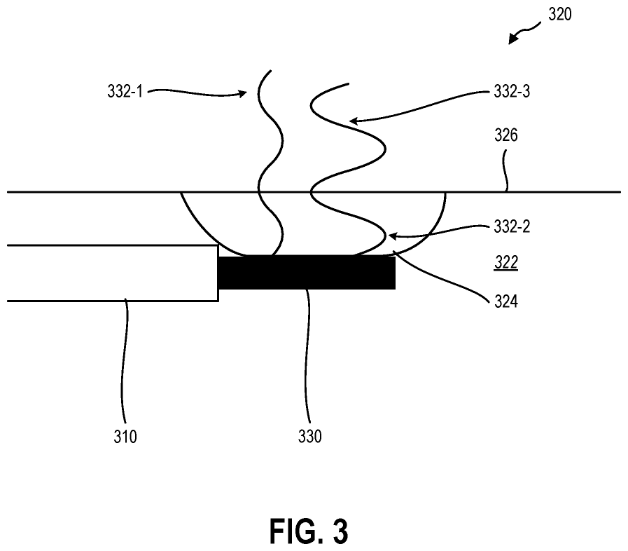

FIG. 3 is a schematic representation of a plaque-removal system 320, closed in on an interface between a reflector 330 and a plaque deposit 324, according to at least one embodiment of the present disclosure. In the embodiment shown, the plaque deposit 324 is attached to a vessel wall 326. The reflector 330 is connected to distal end of a catheter 310 inserted into a body lumen 322 of a vessel proximate the plaque deposit 324. In the embodiment shown, the reflector 330 is in contact with the plaque deposit 324. For example, the reflector 330 is shown in contact with a radially inward portion of the plaque deposit 324. In some embodiments, the reflector 330 may be in contact with any portion of the plaque deposit 324, including a longitudinal edge of the plaque deposit 324.

In accordance with at least one embodiment of the present disclosure, an incoming ultrasound energy 332-1 may be directed to the reflector 330. The reflector 330 may have a high acoustic impedance mismatch with the plaque deposit 324. This may cause at least a portion of the incoming ultrasound energy 332-1 to be reflected as reflected ultrasound energy 332-2. The reflected ultrasound energy 332-2 may combine with at least a portion of the incoming ultrasound energy 332-1 to form combined ultrasound energy 332-3.

In the embodiment shown, the incoming ultrasound energy 332-1 has an inbound amplitude. The inbound amplitude may be representative of the energy emitted by the acoustic energy source. As discussed herein, the acoustic energy source may be located outside of the patient's body. The reflected ultrasound energy 332-2 may have the same amplitude. The reflected ultrasound energy 332-2 may combine with at least a portion of the incoming ultrasound energy 332-1 to form the combined ultrasound energy 332-3. The combined ultrasound energy 332-3 may have a combined amplitude. The combined amplitude may be larger than both the incoming ultrasound energy 332-1 and the reflected ultrasound energy 332-2.

The combined ultrasound energy 332-3 may combine in the plaque deposit 324. In this manner, the ultrasound energy that is applied to the plaque deposit 324 may be greater than the ultrasound energy emitted by the acoustic energy source. Reducing the ultrasound energy emitted by the acoustic energy source may reduce the energy that is applied to other tissues of the user's body. This may help to reduce or prevent damage to the patient from the emitted ultrasound energy that reaches locations other than the plaque deposit 324.

In accordance with at least one embodiment of the present disclosure, the reflector 330 may be placed into contact with the plaque deposit 324 by the catheter 310. The reflector 330 may be placed into contact with the plaque deposit 324 in any manner. The operator of the catheter 310 may axially place the reflector 330 in contact with the plaque deposit 324 by extending or retracting the catheter 310 in the body lumen 322. In some embodiments, the operator of the catheter may use imaging to identify the location of the catheter 310 and the reflector 330.

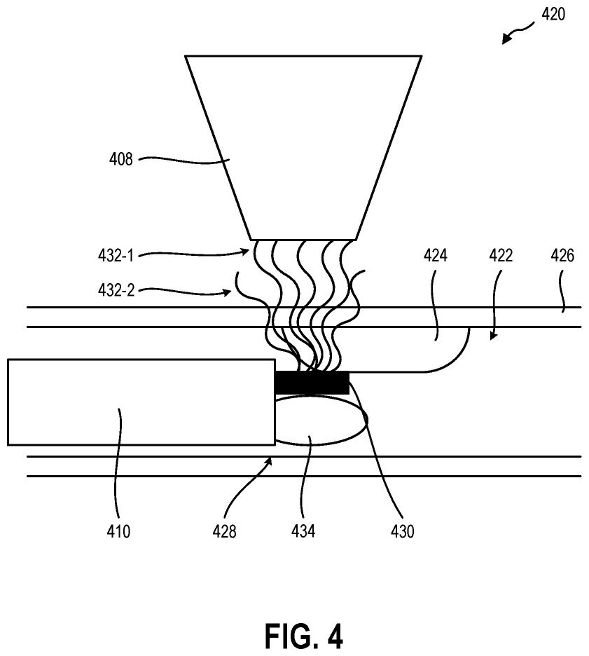

FIG. 4 is a representation of a plaque-removal system 420, according to at least one embodiment of the present disclosure. The plaque-removal system 420 may include a reflector 430 in contact with a plaque deposit 424 on a vessel wall 426 of a patient's body. The reflector 430 is connected to a distal end 428 of a catheter 410 inserted into a body lumen 422 in the vessel walls 426. An acoustic energy source 408 may emit ultrasonic energy (collectively 432) at the reflector 430. An incoming ultrasound energy 432-1 may engage the reflector 430 and be reflected as a reflected ultrasound energy 432-2 to combine to a combined ultrasound energy in the plaque deposit 424.

In accordance with at least one embodiment of the present disclosure, the reflector 430 may be placed in contact with the plaque deposit 424. In the embodiment shown, the reflector 430 may be placed in contact with the plaque deposit 424 by an inflatable element 434. To place the reflector 430 in contact with the plaque deposit 424, the operator may locate the reflector 430 longitudinally proximate the plaque deposit 424. When the reflector 430 is located longitudinally proximate the plaque deposit 424, the operator may cause the inflatable element 434 to be inflated (e.g., the operator may inflate the inflatable element 434). For example, the operator may cause a fluid to be passed through the catheter 410 and into the inflatable element 434. Fluid pressure may cause the inflatable element 434 to inflate. In some embodiments, the inflatable element 434 may be formed from a shape-memory alloy. Cold fluid may be passed through the inflatable element 434 to retain the inflatable element 434 in the retracted or deflated state. To inflate the inflatable element 434, warm fluid may be passed through the inflatable element 434. This may cause the shape-memory alloy to change in shape, thereby causing the inflatable element 434 to inflate.

Inflating the inflatable element 434 may cause the inflatable element 434 to be pushed against the plaque deposit 424. In some embodiments, the reflector 430 may be rotationally oriented such that, when the inflatable element 434 is inflated, the reflector 430 may be placed in contact with the plaque deposit 424.

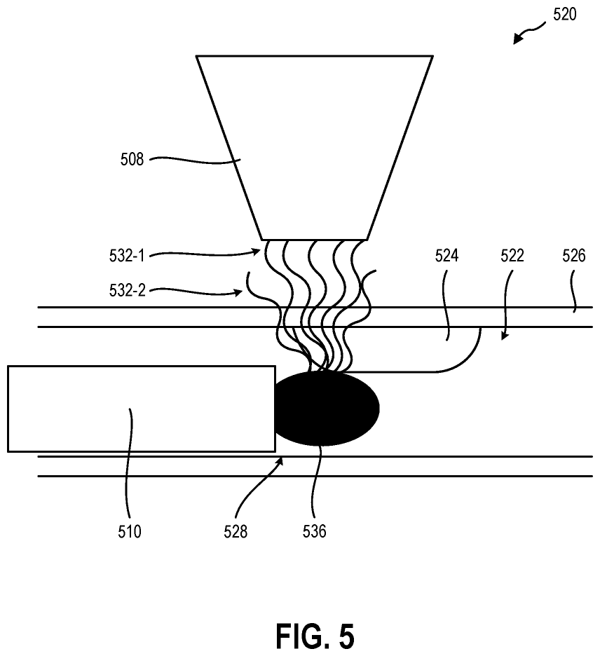

FIG. 5 is a representation of a plaque-removal system 520, according to at least one embodiment of the present disclosure. The plaque-removal system 520 may include an inflatable reflector 536 in contact with a plaque deposit 524 on a vessel wall 526 of a patient's body. The inflatable reflector 536 is connected to a distal end 528 of a catheter 510 inserted into a body lumen 522 in the vessel walls 526. An acoustic energy source 508 may emit ultrasonic energy (collectively 532) at the inflatable reflector 536. An incoming ultrasound energy 532-1 may engage the inflatable reflector 536 and be reflected as a reflected ultrasound energy 532-2 to combine to a combined ultrasound energy in the plaque deposit 524.

In accordance with at least one embodiment of the present disclosure, the inflatable reflector 536 may be placed in contact with the plaque deposit 524. In the embodiment shown, the inflatable reflector 536 may be placed in contact with the plaque deposit 524 by inflating the inflatable reflector 536. For example, the inflatable reflector 536 may be formed from an inflatable element. In some embodiments, the inflatable element may be formed from a material having a high acoustic impedance mismatch with the plaque deposit 524. In some embodiments, the inflatable element may be coated with a reflective coating having a high acoustic impedance mismatch with the plaque deposit 524. In some embodiments, the inflatable element may include a mesh surrounding the inflatable element formed from a material having a high acoustic impedance mismatch with the plaque deposit 524.

To place the inflatable reflector 536 in contact with the plaque deposit 524, the operator may locate the inflatable reflector 536 longitudinally proximate the plaque deposit 524. When the inflatable reflector 536 is located longitudinally proximate the plaque deposit 524, the operator may cause the inflatable reflector 536 to be inflated. For example, the operator may cause a fluid to be passed through the catheter 510 and into the inflatable reflector 536. Fluid pressure may cause the inflatable reflector 536 to inflate and move into contact with the plaque deposit 524. In some embodiments, the inflatable reflector 536 may be formed from a shape-memory alloy. Cold fluid may be passed through the inflatable reflector 536 to retain the inflatable reflector 536 in the retracted or deflated state. To inflate the inflatable reflector 536, warm fluid may be passed through the inflatable reflector 536. This may cause the shape-memory alloy to change in shape, thereby causing the inflatable reflector 536 to inflate, which may place at least a portion of the inflatable reflector 536 in contact with the plaque deposit 524.

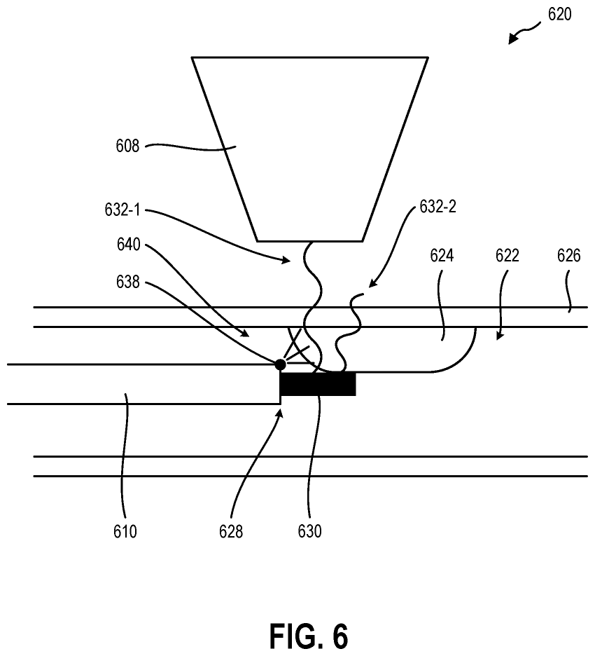

FIG. 6 is a representation of a plaque-removal system 620, according to at least one embodiment of the present disclosure. The plaque-removal system 620 may include a reflector 630 in contact with a plaque deposit 624 on a vessel wall 626 of a patient's body. The reflector 630 is connected to a distal end 628 of a catheter 610 inserted into a body lumen 622 in the vessel walls 626. An acoustic energy source 608 may emit ultrasonic energy (collectively 632) at the reflector 630. An incoming ultrasound energy 632-1 may engage the reflector 630 and be reflected as a reflected ultrasound energy 632-2 to combine to a combined ultrasound energy in the plaque deposit 624.

In accordance with at least one embodiment of the present disclosure, the catheter 610 may include an emission end 638 of the optical energy source. The emission end 638 may emit optical energy 640 directed to the plaque deposit 624. As discussed herein, the optical energy 640 may be applied in conjunction with the ultrasound energy 632. Applying the optical energy 640 with the ultrasound energy 632 may help to remove the plaque deposit 624. As discussed herein, the total energy applied to the patient's body may be reduced using the reflector 630. Because the reflector 630 may reflect at least a portion of the incoming ultrasound energy 632-1 into the plaque deposit 624, the energy level of the incoming ultrasound energy 632-1 may be reduced. This may reduce the total energy applied to the patient, which may help to reduce or prevent injury to the patient.

In some embodiments, the emission end 638 may be located at the distal end 628 of the catheter 610. In some embodiments, the emission end 638 may be located at the reflector 630. In some embodiments, the emission end 638 may include a fiber optic cable that is at least partially connected to the reflector 630. In some embodiments, the emission end 638 may include a fiber optic cable that is at least partially embedded in the reflector 630. In some embodiments, the emission end 638 may include a plurality of optical pathways embedded in the reflector 630 to direct the optical energy to the plaque deposit 624.

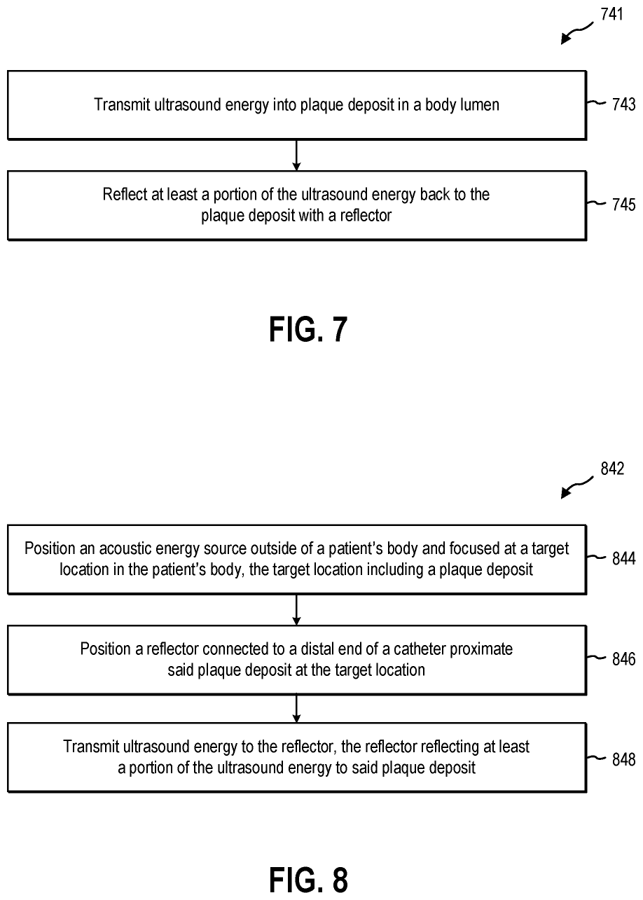

FIG. 7-8, the corresponding text, and the examples provide a number of different methods, systems, devices, and non-transitory computer-readable media of the plaque-removal system 220. In addition to the foregoing, one or more embodiments can also be described in terms of flowcharts comprising acts for accomplishing a particular result, as shown in FIG. 7-8. FIG. 7-8 may be performed with more or fewer acts. Further, the acts may be performed in differing orders. Additionally, the acts described herein may be repeated or performed in parallel with one another or parallel with different instances of the same or similar acts.

As mentioned, FIG. 7 illustrates a flowchart of a method 741 or a series of acts for removing plaque in accordance with one or more embodiments. While FIG. 7 illustrates acts according to one embodiment, alternative embodiments may omit, add to, reorder, and/or modify any of the acts shown in FIG. 7. The acts of FIG. 7 can be performed as part of a method. Alternatively, a computer-readable medium can comprise instructions that, when executed by one or more processors, cause a computing device to perform the acts of FIG. 7. In some embodiments, a system can perform the acts of FIG. 7.

A health care provider may transmit ultrasound energy into a plaque deposit in a body lumen of a patent at 743. For example, the health care provider may orient a HIFU transducer toward the plaque deposit. The HIFU transducer may emit ultrasound energy that is focused on the plaque deposit. In some embodiment, at least a portion of the ultrasound energy may be reflected back toward the plaque deposit with a reflector at 745. The reflector may be located at a distal end of a catheter located at the plaque deposit in the body lumen.

In some embodiments, the ultrasound energy may be transmitted at the reflector when the reflector is in contact with the plaque deposit. In some embodiments, the ultrasound energy may be placed in contact with the plaque deposit by inflating an inflatable element.

In some embodiments, the operator may provide a sequence of laser pulses through an optical conduit in the catheter. In some embodiments, providing the sequence of laser pulses may occur simultaneously while transmitting the ultrasound energy. In this manner, the ultrasound energy and the optical energy may combine to remove at least a portion of the plaque deposit.

As mentioned, FIG. 8 illustrates a flowchart of a method 842 or a series of acts for removing plaque in accordance with one or more embodiments. While FIG. 8 illustrates acts according to one embodiment, alternative embodiments may omit, add to, reorder, and/or modify any of the acts shown in FIG. 8. The acts of FIG. 8 can be performed as part of a method. Alternatively, a computer-readable medium can comprise instructions that, when executed by one or more processors, cause a computing device to perform the acts of FIG. 8. In some embodiments, a system can perform the acts of FIG. 8.

In accordance with at least one embodiment of the present disclosure, an acoustic energy source, such as a HIFU transducer, may be placed outside of a patient's body and focused at a target location in the patient's body at 844. The target location may include a target tissue, such as a plaque deposit or other tissue. A reflector connected to a distal end of a catheter may be placed proximate the plaque deposit at the target location at 846. For example, the reflector may be connected to the distal end of the catheter. A health care provider may insert the catheter into a vessel in the patient's body, such as an artery or vein. The health care provider may direct the distal end of the catheter, including the reflector, to the target location. In some embodiments, the heath care provider may cause the reflector to contact the plaque deposit. For example, as discussed herein, the health care provider may cause an inflatable element to inflate to place the reflector in contact with the plaque deposit. As discussed herein, placing the inflatable element in contact with the plaque deposit may occur by inflating an inflatable element at the distal end of the catheter.

In some embodiments, the health care provider may transmit ultrasound energy to the reflector using the HIFU transducer at 848. The reflector may reflect at least a portion of the ultrasound energy to the plaque deposit. The reflected energy may combine with the incoming ultrasound energy to increase the total energy applied to the plaque deposit. This may help to reduce the total energy emitted by the HIFU transducer. As discussed herein, the health care provider may provide a laser pulse to the plaque deposit through an optical conduit in the catheter. In some embodiments, the reflector may reflect all of the ultrasound energy back toward the plaque deposit.

In some embodiments, the HIFU transmitter may be positioned such that the plaque deposit is located between the reflector and the HIFU transmitter.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A system, comprising:
an ultrasound transducer;
a catheter for insertion into a body lumen of a patient, the catheter including a distal end; and
a reflector connected to the distal end of the catheter, the reflector formed from a material having an acoustic impedance mismatch with an adjacent material.

2. The system of claim 1, wherein the ultrasound transducer is separate from the catheter.

3. The system of claim 2, wherein the ultrasound transducer is configured to be operated outside of said body lumen.

4. The system of claim 1, further comprising an optical energy source connected to the catheter, an emission end of the optical energy source located at the distal end of the catheter.

5. The system of claim 4, wherein the emission end is at least partially embedded in the reflector.

6. The system of claim 1, further comprising an inflatable element proximate the reflector at the distal end of the catheter.

7. The system of claim 6, wherein the inflatable element includes the reflector.

8. The system of claim 1, wherein the reflector is formed from a steel alloy.

9. The system of claim 1, wherein the reflector is located between a plaque deposit and the ultrasound transducer.

10. A method, comprising:
transmitting ultrasound energy into a plaque deposit in a body lumen; and
reflecting at least a portion of the ultrasound energy back to said plaque deposit with a reflector, the reflector connected to a distal end of a catheter located at said plaque deposit in said body lumen.

11. The method of claim 10, wherein transmitting the ultrasound energy includes transmitting the ultrasound energy at the reflector while the reflector is in contact with said plaque deposit.

12. The method of claim 11, further comprising inflating an inflatable element to place the reflector in contact with said plaque deposit.

13. The method of claim 10, further comprising providing a sequence of laser pulses through an optical conduit to said plaque deposit.

14. The method of claim 13, wherein providing the sequence of laser pulses occurs simultaneously with transmitting the ultrasound energy.

15. A method, comprising:
positioning an acoustic energy source outside of a patient's body and focused at a target location in said patient's body, the target location including a plaque deposit;
positioning a reflector connected to a distal end of a catheter proximate said plaque deposit at the target location; and
transmitting ultrasound energy to the reflector, the reflector reflecting at least a portion of the ultrasound energy to said plaque deposit.

16. The method of claim 15, further comprising placing the reflector in contact with said plaque deposit, and wherein transmitting the ultrasound energy includes transmitting the ultrasound energy while the reflector is in contact with said plaque deposit.

17. The method of claim 16, wherein placing the reflector in contact with said plaque deposit includes inflating an inflatable element to place the reflector in contact with said plaque deposit.

18. The method of claim 15, further comprising providing a laser pulse through an optical conduit to said plaque deposit at the target location.

19. The method of claim 15, wherein positioning the acoustic energy source includes positioning the acoustic energy source such that said plaque deposit is located between the reflector and the acoustic energy source.

20. The method of claim 15, wherein the reflector reflects all of the ultrasound energy to said plaque deposit.
